(19) United States
(12) Patent Application Publication
(10) Pub. No.: US 2022/0299431 A1
(43) Pub. Date: Sep. 22, 2022

(54) ASSAY MEMBRANE TEST REGION LOCALIZATION

(71) Applicant: Kenota Inc., Kitchener (CA)

(72) Inventors: Christopher J. Harder, Elmira (CA); Yiheng Qin, Mississauga (CA)

(21) Appl. No.: 17/698,953

(22) Filed: Mar. 18, 2022

Related U.S. Application Data
(60) Provisional application No. 63/163,529, filed on Mar. 19, 2021.

(51) Int. Cl. G01N 21/33; G01N 21/77; G01N 21/84; G01N 33/52; G01N 33/543; G01N 33/558; G01N 35/00

(52) U.S. Cl. CPC G01N 21/33; G01N 21/77; G01N 21/8483; G01N 33/523; G01N 33/54388; G01N 33/558; G01N 35/00693; G01N 2021/1765

(57) ABSTRACT

A method for localizing a test region of interest on an assay membrane to determine the contours of the test region and enable calibration of the location of the test region such that the same region can be localized to image an analyte of interest after an assay run. Pre-localization of the test region limits the contours of the detection area to only the test region with a reasonable margin such that background noise received by the detector can be minimized. By limiting the region of detection to a pre-localized test region improved accuracy can be achieved in flow assay membrane tests, in particular in automated analyzer systems.

### CROSS-REFERENCE TO RELATED APPLICATIONS

This application claims priority to United States provisional patent application U.S. 63/163,529 filed 19 Mar. 2021, which is hereby incorporated by reference herein in its entirety.

### FIELD OF THE INVENTION

The present invention is directed to a method and device for increasing accuracy of flow assay membrane test results using labeling and localization of test regions. The present invention is also directed to localization of test and control regions of interest on an assay membrane where there are spatially separated signal detection regions in a results area.

### BACKGROUND

Analytical analyte binding assays are useful in diagnostic applications, for example, in human health, environmental assessment, and industrial food and drug preparation. Lateral flow membrane assays, being one type of these binding assays, are based on the principles of immunochromatography and exist for a wide array of target analytes. Assay membranes are commercially available for many applications including monitoring ovulation, detecting infectious disease organisms, analyzing drugs of abuse, and measuring other analytes important to human physiology, as well as for veterinary testing, agricultural applications, environmental testing, and product quality evaluation. While the assay membrane tests provide qualitative results based on the presence or absence of a signal line in a test area, lateral flow assay test design has progressed toward semiquantitative and quantitative assays with the integration of hand-held readers and high throughput analyzers.

Most lateral flow assay membranes are modeled after existing immunoassay formats and are typically sandwich assays in which an antigen or molecule of interest is immobilized between two layers of antibodies, a capture antibody immobilized at a test region and a mobile detection antibody having a bound detectable species. Other analyte binding assays, including immunoassays, utilize a broad range of test formats, such as agglutination assays, precipitin assays, enzyme-linked immunoassays, direct fluorescence assays, immuno-histological tests, complement-fixation assays, serological tests, immuno-electrophoretic assays, and lateral flow and flow through tests. In blood-based assays, proteins and other molecules can be detected as indicators of various disease states and immunological status, and can detect the formation of one or more complexes between a detector particle that is free in the sample stream and a capture reagent or immobilized binding species that is bound to the membrane at a test region of interest.

The ability to obtain meaningful and accurate results in analyte binding assays using smaller sample volumes is important when testing samples that are difficult to acquire in large volume, such as point-of-care tests for human health. As the size of test devices decreases and the sample test volume decreases, detection methods for determining the presence or absence of a species of interest requires increased sensitivity compared to inspection methods, especially when the number of analytes of interest detected on a single assay membrane is high and/or when the concentration of analyte of interest in the sample is low. In the use of automated analyzers or point-of-care devices, ensuring accurate results during high throughput testing is critical to having reasonable confidence in the results of an assay membrane test. In addition, quantitation of results is increasingly being used to glean more information from tested samples, putting yet a greater burden on the accuracy requirements for automated detection systems.

In the manufacture of assay membranes there can be slight but significant variation in the location as well as the concentration of species applied to the membrane which can affect the results of the assay. Visualization of test and control areas using automated visualization can assist in improving accuracy of test results. In one example, U.S. Pat. No. 10,254,232 to Yoo et al. describes a device and method for detecting an analyzed object in a specimen by comparing the reflectance signals before and after a lateral flow test is run. In this method, the background area, control area, and test area of a membrane is illuminated by two different illumination light sources, and the light emitted from the test area, the control area, and the background area, respectively, is detected by each of the light receiving units to calibrate the background noise.

In an example of lot-to-lot calibration of assay membranes, U.S. Pat. No. 9,671,401 to Irvin provides a method of adjusting a final signal value measured on a lateral flow assay test strip by adjusting the reflectance value measured on a test strip to compensate for variations in results exhibited among similar test strips to adjust the final measured reflectance value by comparison to test results exhibited by other test strips from the same manufacturing lot.

In high throughput automated analyzers, misalignment of the test and control areas as well as variation in concentration of species applied to the membrane can result in variable and therefore inaccurate interpretation of the assay results. There remains a need for improving detection and quantitation of species on assay membranes, in particular when used in an automated assay membrane analyzing device.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a method of detection of species of interest from a sample using an analyte binding assay. It is another object of the present invention to provide a method for localizing a test region of interest on an assay membrane using a non-interfering localization label. It is another object of the present invention to provide a method and system for pre-labeling and pre-localization of test and control regions in areas on an assay membrane prior to an assay run such that detection at the same location can be done after the assay run. Control and test region localization provides more accurate automated signal detection by reducing the detection area and minimizing background noise in the detection of signal at the region of interest, in particular in the use of automated signal detection systems. The present invention has also been found to reduce background noise during assay results detection.

In an aspect of the present invention there is provided a method for localizing an analyte of interest on a test region of an assay membrane comprising: imaging a localization species in the test region, the localization species having a molecular property that, upon imaging, differentiates the test region from a background of the assay membrane; determining contours of the test region by imaging the localization label and the background around the region of interest and comparing intensity of the background of the assay membrane to intensity at the region of interest; and imaging an analyte of interest inside the contours of the test region after exposing the assay membrane to a running buffer to run the assay, the analyte of interest bound to a detectable analyte label and an immobilized binding species at the test region.

In an embodiment of the method, imaging the localization label in the test region is performed prior to running the assay, and further comprising, before imaging the analyte of interest: applying a sample comprising the analyte of interest to the assay membrane; and applying a running buffer to the assay membrane to run the assay.

In an embodiment of the method, the assay membrane further comprises at least one control region of interest, the control region of interest comprising localization label and an additional immobilized binding species.

In another embodiment of the method, the localization label is an organic dye, inorganic dye, fluorescent molecule, phosphorescent molecule, radiating molecule, or colored bead.

In another embodiment of the method, the localization label is brilliant blue FCF, prussian blue, quinoline yellow WS, gold nanoparticles, europium nanoparticles, Cu doped zinc sulfide, glass beads, carbon nanotubes, HgTe quantum dots, phthalocyanine, or a combination thereof.

In another embodiment of the method, wherein the localization label or the immobilized detection species is conjugated with monoclonal anti-human IgE.

In another embodiment of the method, pre-localization imaging comprises exposing the test region of interest to an external stimulus to image a contrast between the localization label and the background.

In another embodiment of the method, the external stimulus is white light or ultraviolet light.

In another embodiment of the method, the localization label comprises a fluorescent species, and the external stimulus comprises a light source in an absorbance band of the fluorescent species.

In another embodiment of the method, the molecular property of the localization label is wavelength, frequency, phase, amplitude, intensity, delay time, energy, fluorescence lifetime, refractive index, reflectance, absorbance, emissivity, transmittance, polarization, dispersion, scattering, or a combination thereof.

In another embodiment of the method, the localization label is free flowing and washed away from the test region of interest by the running buffer during the assay run.

In another embodiment, the localization label is applied to the test region before manufacturing, and the localization label is soluble in the running buffer and washed away from the test region during the assay.

In another embodiment of the method, the localization label on the assay membrane is in an amount proportional to the immobilized binding species at the region of interest in a proportionality constant.

In another embodiment, the method further comprises using the proportionality constant to calculate a concentration of analyte of interest in the sample.

In another embodiment, the method further comprises housing the assay membrane in a cartridge.

In another embodiment of the method, the assay membrane is a lateral flow assay membrane.

In another embodiment, the method is carried out in an automated analyzer.

In another aspect there is provided a method for identifying a region of interest on an assay membrane comprising: pre-localizing a region of interest on an assay membrane, the region of interest comprising a localization label and an immobilized binding species, the localization label having a molecular property that, upon imaging, differentiates the region of interest from a background of the assay membrane; determining contours of the region of interest by imaging the localization label and the background around the region of interest and comparing intensity of the background of the assay membrane to intensity at the region of interest; applying a sample comprising an analyte of interest to the assay membrane; applying a running buffer to the assay membrane to run the assay; and after the assay run, imaging the pre-localized region of interest to detect binding of the analyte of interest to the immobilized binding species, wherein signal from the analyte of interest bound to the immobilized binding species is inside the contours of the region of interest.

In another aspect there is provided a method for manufacturing an assay membrane comprising: applying a localization label to a test region of interest on an assay membrane, the localization label having a molecular property that, upon imaging, differentiates the test region from a background of the assay membrane; and applying an immobilized binding species to the test region on the assay membrane, wherein the localization label does not interfere with binding of the immobilized binding species to an analyte of interest during an assay run.

In an embodiment of the method, the localization label is soluble in assay running buffer.

In another embodiment of the method, the assay membrane is a lateral flow assay membrane.

In another embodiment, the method further comprises mixing the localization label and the immobilized binding species in a test solution and applying the test solution to the assay membrane during manufacturing.

In another embodiment of the method, the localization label and the immobilized binding species are present in a known ratio at the region of interest.

In another aspect there is provided a method for detecting an analyte of interest on an assay membrane comprising: providing a lateral flow assay membrane with a sample addition area and a results area downstream the sample addition area, the results area comprising at least one test region and at least one control region, the test region and the control region each comprising an immobilized binding species and an immobilized localization label; applying a sample comprising an analyte of interest to the sample addition area; applying running buffer to run the assay; visualizing the test region and control region with an imaging system and a first imaging modality that locates the immobilized localization label at the test region and the control region to identify binding regions of interest; and visualizing the test region and control region with a second imaging system and a second imaging modality at the identified binding regions of interest, the immobilized localization label having a molecular property that differentiates a test region of interest around the test region and a control region of interest around the control region from background.

In an embodiment, the method further comprises determining contours of the region of interest by imaging the localization label and the background around the region of interest and comparing intensity of the background of the assay membrane to intensity at the region of interest.

In another aspect there is provided a lateral flow assay device comprising: a sample addition area; a results area downstream the sample addition area comprising at least one test region and at least one control region, the test region and the control region each comprising an immobilized binding species and a localization label, the localization label having a molecular property that, upon imaging, differentiates a region of interest around the test region and a region of interest around the control region from a background in the results area.

In another aspect there is provided a lateral flow assay device comprising: a sample addition area; a results area downstream the sample addition area comprising at least one test region and at least one control region, the test region comprising an immobilized binding species and a localization label, the localization label having a molecular property that, upon imaging prior to assay run, differentiates a region of interest around the test region from a background in the results area.

In an embodiment of the device, the localization label on the test region is in an amount proportional to the immobilized binding species.

In another embodiment of the device the localization label is soluble in assay running buffer and washed away from the results area by the running buffer during the assay run.

In another embodiment of the device the molecular property of the localization label is one or more of wavelength, color, frequency, phase, amplitude, intensity, delay time, energy, fluorescence lifetime, refractive index, reflectance, absorbance, emissivity, transmittance, polarization, dispersion, and scattering.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 is an isometric view of an example assay membrane;

FIG. 2A is an illustration of the results area of an example assay membrane;

FIG. 2B is an illustration of one test region in a region of interest on an assay membrane;

FIG. 3 illustrates a method for localization of regions of interest on an assay membrane;

FIG. 4 is an example high signal result from a lateral flow assay membrane assay using a pre-localization method;

FIG. 5 is an example low signal result from a lateral flow assay membrane assay using a pre-localization method;

FIG. 6 is a flowchart of a method for pre-localization of a region of interest on an assay membrane;

FIG. 7 is a flowchart of a visualization method for pre-localization a region of interest on an assay membrane;

FIG. 8 is an illustration of a flow assay membrane with a localization label after manufacturing, before an assay run, and after an assay run;

FIG. 9 is a flowchart of a method for detection of signal at a region of interest after localization and run of the assay;

FIG. 10 is a flowchart of a method for manufacturing an assay membrane with a localization label for pre-localization of a region of interest;

FIG. 11 is a panel of assay membranes with regions of interest during a pre-localization and post-localization method;

FIG. 12A is a flowchart of a method for test region localization on an assay membrane using a pre-run localization of the region of interest;

FIG. 12B is a flowchart of a method for test region localization on an assay membrane using more than one imaging modalities in a post-run localization method;

FIG. 13 shows an assay membrane test region pre-localization with an assay membrane having a mobile localization label pre-applied to the test region;

FIG. 14 shows an assay membrane test region pre-localization with an assay membrane having a non-mobile localization label pre-applied to the test region; and

FIG. 15 shows an assay membrane test region localization with an assay membrane having a localization label binding species pre-applied to the test region and a localization label in the assay running buffer.

### DETAILED DESCRIPTION OF THE INVENTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

As used herein, the terms “comprising,” “having,” “including,” and “containing,” and grammatical variations thereof, are inclusive or open-ended and do not exclude additional, unrecited elements, features, and/or method steps. These terms, when used herein in connection with a composition, device, article, system, use, or method, denote that additional elements, features, and/or method steps may be present. A composition, device, article, system, use, or method described herein as comprising certain elements and/or steps may also, in certain embodiments consist essentially of those elements and/or steps, and in other embodiments consist of those elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to. The recitation of ranges herein is intended to convey both the ranges and individual values falling within the ranges, to the same place value as the numerals used to denote the range, unless otherwise indicated herein.

The use of any examples or exemplary language, e.g. “such as”, “exemplary embodiment”, “illustrative embodiment” and “for example” is intended to illustrate or denote aspects, embodiments, variations, elements or features relating to the invention and not intended to limit the scope of the invention.

As used herein, the terms “connect” and “connected” refer to any direct or indirect physical association between elements or features of the present disclosure. Accordingly, these terms may be understood to denote elements or features that are partly or completely contained within one another, attached, coupled to, disposed on, joined together, in communication with, operatively associated with, or fluidically coupled to, etc., even if there are other elements or features intervening between the elements or features described as being connected.

The term “sample” as used herein, refers to a volume of a liquid, fluid, solution, or suspension, intended to be subjected to qualitative or quantitative determination of any of its properties or components, such as the presence or absence of a component, the concentration of a component, etc. Typical samples used in the context of the present invention as described herein are biological or chemical samples derived from human or animal bodily fluids such as but not limited to blood, plasma, serum, lymph, urine, saliva, semen, amniotic fluid, gastric fluid, phlegm, sputum, mucus, tears, stool, etc. Other types of samples that can be used with the present invention can be derived from human or animal tissue samples where the tissue sample has been processed into a liquid, solution, or suspension to reveal particular tissue components for examination. Other non-limiting examples of samples that can be used are environmental samples, food industry samples, and agricultural samples.

The terms “analyte,” “analyte of interest,” and “species of interest” in this disclosure refer to any and all clinically, diagnostically, or relevant chemical or biological analytes present in a sample. Analytes of interest can include, but are not limited to antibodies, hormones, molecules, antigens, organic chemicals, biochemicals, and proteins. Some non-limiting examples of antibodies include antibodies that bind food antigens, and antibodies that bind infectious agents such as virus and bacteria, for example anti-CCP, anti-streptolysin-O, anti-HIV, anti-hepatitis (anti-HBc, anti-HBs etc), antibodies against Borrelia, and specific antibodies against microbial proteins.

The term “analyzer” as used herein, refers to any apparatus enabling the automated processing of one or multiple analytical test assay membranes, and in which a plurality of assay membrane test devices may be processed. The analyzer can comprise a plurality of components configured for, for example, loading, incubating, testing, transporting, imaging, and evaluating a plurality of analytical test elements in an automated or semi-automated fashion, and in which sample and/or other fluids may be automatically dispensed and processed substantially without user intervention. Analyzers include but are not limited to clinical diagnostic apparatus and point-of-care type devices.

The term “reaction” as used herein, refers to any interaction which takes place between components of a sample and at least one reagent or reagents on or in, or added to, the substrate or membrane of the assay membrane device, or between two or more components present in the sample. The term “reaction” is used to define the interaction taking place between an analyte and a reagent on the test device as part of the qualitative or quantitative determination of the analyte. The term “reaction” also includes but is not limited to reversible or irreversible binding of two or more molecules, one of which is usually the analyte of interest.

The term “region of interest” and the acronym “ROI” as used herein refer to a region on the assay membrane where a bound or immobilized species is localized. The region of interest can comprise one or more antibodies, antigens, detection agents, conjugated antibodies, tagging molecules, fluorophores, biomarker specific antibodies, DNA molecules, RNA molecules, aptamers, or probes, that independently or together with another molecule, are capable of binding to a species of interest in the sample that the assay membrane is designed to detect. The term region of interest is also used in context of a “results area” where a broader “result area” would include one or more “regions of interest”.

The terms “localization label” and “localization species” as used herein refers to any species on or applied to an assay membrane that can be detected by a detector in advance of the assay run to determine the location of a region of interest. The localization label can be bound to the region of interest, also referred to herein as ‘immobile’, or unbound or mobile such that it flows away from the region of interest during running of the assay after the addition of a running buffer. The localization label can also be the same or different from the reporter, also referred to as the immobilized binding molecule, which binds to the analyte of interest.

The term “running buffer” as used herein refers to a solution, also referred to as mobile fluid or developing solution, which is applied to the sample addition area of a flow assay membrane to perform the assay. In a lateral flow assay the running buffer flows along the fluid flow path toward the reaction area or detection area on the assay membrane. The running buffer can contain the sample or be separate from the sample prior to application to the membrane. The running buffer is preferably aqueous and comprises one or more buffers, salts, and detergents.

Herein is provided a method of increasing precision of a lateral flow test assay using pre-localization of one or more region of interest on an assay membrane using an automated analyzer and imaging. Pre-localization of test regions on an assay membrane enables calibration of the location of the test region(s) of interest on the assay membrane such that the same region(s) can be localized after the assay has been run to detect the presence of an analyte of interest. Detection in the region(s) of interest after the assay has been run limits the detection region to only the region(s) of interest with a reasonable margin such that background noise received by the detector in the analyzer can be minimized. By limiting the region of detection after the assay run to the pre-localized region(s) of interest improved accuracy can be achieved, especially in automated analyzer systems. For quantitative automated high throughput lateral flow assay analysis reliable enough to replace laboratory results, an accurate and precise method of calibration must exist to get similar results as a laboratory. Pre-localization of regions of interest on the assay membrane has been found to reduce the background noise captured by the optical detection system and provide a broader range of binding signal, resulting in robust and reliable automated assay results.

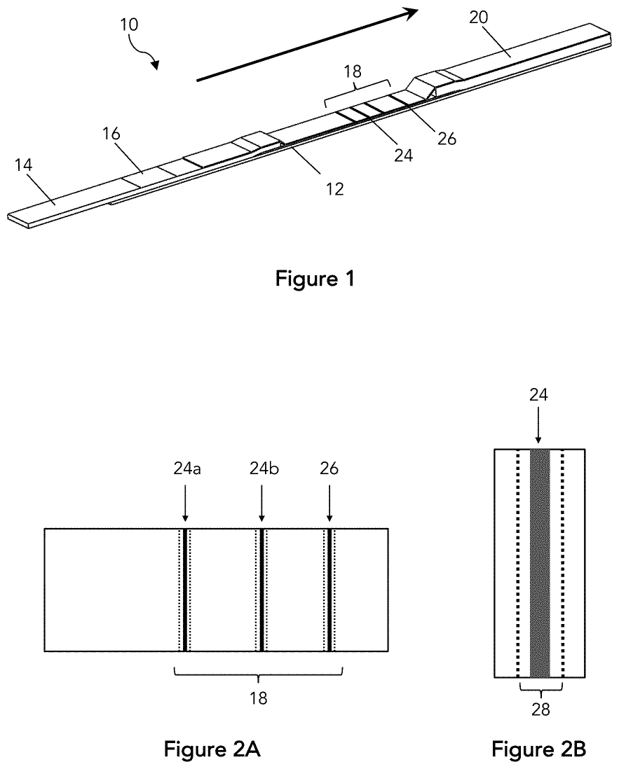

FIG. 1 is an isometric view of an example flow assay membrane 10 which can be used with the present method as a diagnostic test device. The assay membrane 10 comprises, in series along a flow path: a sample addition area 16; a results area 18 comprising a test region 24 and a control region 26, each of the test region and the control region having an immobilized binding molecule or species; and a wicking area 20. A sample addition area 16 at the upstream side of the lateral flow assay membrane 10 extends through one or more fluidly connected membrane to a results area 18 comprising the test region 24 and control region 26, and a wicking area 20. The arrow shows the direction of flow of running buffer, also referred to as the fluid flow path. The assay membrane 10 is preferably encased in a cartridge for protection and handling of the assay membrane in an automated analyzer.

A localization label is a molecule that is used in the assay to locate the test region 24. The localization label can be detected using a first imaging modality either before or after the assay run to determine the contours of the test region 24. After sample and running buffer are added to the assay membrane, an analyte label can be detected using the same or different imaging modality, where the analyte label binds an analyte of interest in the sample and to an immobilized binding species in the test region 24. By localizing the region of interest at the test region 24 using a localization label the imaging analysis of the analyte label and analyte of interest can be restricted to the known contours of the test region 24.

The localization label is a molecule or marker having a molecular property that is differentiated from the membrane background around it such that it can be located by imaging, optionally with a stimulus, at the region of interest where it has been applied. The assay membrane or fluid applied thereto comprises a detectable species, also referred to as the analyte label, that binds to an analyte of interest in a sample, either directly or through a coupling molecule, to visualize the presence of the analyte of interest by binding with the analyte of interest at one or more regions of interest through an immobilized binding molecule at one or more test regions, test lines, or test spots. The localization label can be the same or different than the analyte label, but does not interfere with the binding of the analyte of interest to the immobilized binding species on the assay membrane. In one assay membrane design the localization label is applied to the test region 24 and is soluble in running buffer such that it is washed away by the running buffer. In another assay membrane design the localization label is immobilized on the test region 24 but does not impede binding and detection of any bound analyte of interest to the immobilized binding species at the region of interest.

The localization label or a localization label binding species that binds the localization label can be positioned inside, outside, or both inside and outside the test regions of interest on the assay membrane and can be positioned above, inside, or beneath any layer of the assay membrane. A localization label is a molecule that is used reversibly or irreversibly bound to the localization or test region and can be pre-localized or bound to the localization region during an assay run. Example of localization labels include but are not limited to dyes or other colorimetric molecules, fluorophores, radio labels, fluorochromes, or any other molecule that produces a signal detectable by an imaging system. The localization label can be unbound to the membrane and free flowing upon addition of running buffer. Alternatively, the localization label can bind to a localization label binding species, and the method of binding of the localization label to the localization label binding species can be, for example, a direct covalent or weaker non-covalent attachment method. Non-covalent methods could include macromolecular anchoring molecules such as but not limited to antibodies, avidin or streptavidin, aptamers, nucleic acid with their appropriate binding pairs. The molecular property of the localization label can, for example, be such that the localization label reflects and/or absorbs and/or emits electromagnetic waves of wavelength between 10 nm to 1 mm. The molecular property of the localization label, in particular the reflection, absorption, and/or emission of electromagnetic waves, can be spontaneous or triggered by an external excitation or stimulus such as, for example, temperature variation, mechanical force, electromagnetic wave, chemical reaction, biochemical reaction, radiation, electron transfer, filtration, polarization, and light splitting.

The localization label can either be mobile or unbound and flow away during the assay run after application of running buffer, or be immobilized on the assay membrane and remain in place during the assay run. A localization label that can flow away with the running buffer ensures that the localization label will not interfere with the post-run molecular signal and with analyte detection. Alternatively, a localization label that does not interfere with the post-run molecular signal can be immobile and can also be used, and the localization of the localization label at the test region 24 can be done in a different imaging modality than that used to detect analyte of interest bound to an analyte label in the same test region 24. Some examples of localization labels which can be used with the present device and method include but are not limited to organic dyes, inorganic dyes, fluorescent molecules, phosphorescent molecules, radiating molecules, and colored beads. Some specific examples of localization labels include Brilliant Blue FCF, Prussian blue, Quinoline Yellow WS, gold nanoparticles, europium nanoparticles conjugated with monoclonal anti-Human IgE, luminol, copper (Cu) doped zinc sulphide (ZnS), glass beads, carbon nanotubes, mercury telluride (HgTe) quantum dots, and phthalocyanine.

The localization label can also be water-soluble species that defines and identifies the region of interest for measuring the signal of a binding species and acts as a proxy for concentration of the analyte in question, and optionally washes away upon assay run and does not interfere with the reporter or analyte label that detects the analyte. In addition, the molecular property of the localization label can be proportional to a concentration of binding agent or immobilized binding species deposited at the test region of interest to bind the analyte of interest, and the molecular property and a proportionality constant can be used to calculate the concentration of the immobilized binding species which binds the analyte of interest at the test region 24. The comparison of the signal intensity of the imaged region of interest pre-labeled with the localization label before the assay run can also provide an indication of the age of the assay membrane, as binding species on the assay membrane can degrade with time and a decreased signal intensity of the pre-localization label can be indicative of an older, damaged, or less sensitive assay membrane. Pre-labeling the assay membrane using a localization label or localization label binding species that binds a mobile localization label and comparing it to signal from the analyte label and analyte of interest bound to the immobilized binding species at a known ratio and knowing the degradation rate of each over time can also provide additional information on the integrity of the assay membrane and offers an opportunity of adjustment of the reported results based on the integrity or age of the assay membrane.

In use, once sufficient fluid is added to the sample addition area 16 on the assay membrane or to an area partially overlapping with sample addition area 16, or upstream of the sample addition area 16 at the optional conjugate pad 14, the sample and running buffer flows along the defined fluid flow path (shown with an arrow) by capillary action between the sample addition area 16 and the wicking area 20. Fluid to run the sample can be a sample fluid, i.e. fluid containing the analyte of interest, running buffer, sample fluid mixed with running buffer, or a small amount of sample fluid followed by a sufficient amount of running buffer to run the assay. The sample addition area 16 on the diagnostic test device refers to the area on the assay membrane where a sample to be analysed is dispensed. Conjugate pad 14 can further comprise one or more conjugate detectable species and can also be used to transfer sample or running buffer upstream of the sample addition area 16 to the assay membrane. The assay membrane is preferably housed in a cartridge housing (not shown) to protect the assay membrane and compounds deposited thereon and to assist with receiving sample and running buffer. An optional solid support 12 can also provide structural support to the components of the assay membrane. The assay cartridge can further have one or more identifier for identifying the assay membrane, such as a barcode or other identifier, which can be any textual or digital data stored as an image that can be read by an optical reader or person. Alternatively, the assay cartridge can have one or more other identification tags such as, for example, an RFID tag or electromagnetic label.

In a typical lateral flow assay, a stationary or bound (immobilized) binding molecule at test region 24 binds to and indicates the presence (or absence) of an analyte of interest, with relative line intensity being correlated with the amount of analyte of interest in the sample applied to the assay membrane. Three test regions are shown as test lines, however the assay membrane can have one or more test regions. The control region 26 also comprises a stationary or bound (immobilized) reporter binding molecule which binds with a reporter molecule once the reporter passes the control region 26 to indicate a valid test. The sample applied to an assay membrane is investigated to determine if it comprises the species of interest by running the assay with a developing solution or running buffer to see if the species of interest is present in the sample by binding to a region of interest at the test region 24 on the assay membrane. Each test and control region comprises a binding species, and in many standard lateral flow assay tests the binding species on the test region and the control region is usually invisible to optical systems prior to developing the assay. By supplementing the applied reagent on the regions of interest at the test region(s) with a localization label that is detectable by a visualization or imaging system, the region of interest on the membrane in the test or results area can be pre-localized. Upon interaction of an immobilized binding species at the region of interest on the assay membrane in the presence of the species or analyte of interest together with an analyte label the assay membrane can be visualized at the pre-localized region(s) of interest at the test region(s) to detect the presence of the species of interest in the sample.

In a standard immunoassay, a conjugate binding molecule binds an analyte of interest, which is then captured by an immobilized capture species at the test region. In the present pre-localization method the detectable species or detection conjugate can be the same or different from the localization label. The lateral flow or assay membrane is referred to herein in terms of the exemplary embodiment shown, however it will be readily apparent that other assay membrane device designs and possible variants of these designs could also be similarly configured for interrelationships with the presently described method and device for sample volume and concentration normalization and control and test region pre-localization in a lateral flow assay, particularly in an automated analyzer system, as herein described. Other assay membrane devices comprising various regions of interest including lines, spots, and comprising various membrane configurations can be used, for example for chromatographic, lateral flow, and enzyme-linked immunosorbent assay (ELISA) type assays.

To run the assay after pre-localization of a test region 24 having a pre-applied mobile localization label, sufficient running buffer is applied either directly to the sample addition area 16 or to the conjugate pad 14 or into a running buffer reservoir in the cartridge housing the assay membrane 10 that is fluidly connected to the sample addition area 16. In the embodiment shown, conjugate pad 14 at the first or upstream front end of the fluid flow path draws sample fluid and/or running buffer in the desired direction along the assay membrane optionally from a reservoir in a cartridge housing the assay membrane and provides a capillary force to draw up and move sample running buffer into the membrane of the assay membrane and through the sample addition area 16. The conjugate pad 14 is preferably composed of a glass fibre to allow mixing, and can optionally also include a porous material such as, for example, nitrocellulose, which can act as a size exclusion membrane and slow fluid flow. Conjugate pad 14 is optionally bendable, shown extending off from an optional solid support 12, to accommodate a lowered buffer well or reservoir in the assay cartridge base and further positioned by an optional wick guide in the assay cartridge base and/or lid to ensure fluid contact with the running buffer. Obvious asymmetry in the design of the assay membrane also provides ease of assembly of the assay membrane within an assay cartridge and provides a directionality of the flow path so that the assay membrane is properly aligned inside the cartridge. Consistent alignment of the assay membrane in the cartridge can also assist with alignment of the cartridge and regions of interest on the assay membrane in the analyzer. Optionally a hydrophilic foil or layer (not shown) can be positioned onto at least a portion of the assay membrane to enhance the overall flow rate or process time of a sample applied to the flow assay membrane. The present membranes can be very small, and example membranes used in this method are 3 mm in width, providing an idea of the scale of the present membrane and its features. It is understood, however, that a variety of assay membrane sizes may be used.

The assay membrane can also optionally comprise one or more flow channels, optionally cut or pressed into the surface of the membrane substrate. The fluid flow path may also include additional separate areas containing one or more reagents, antibodies, or detection conjugates (detectable species), as well other areas or sites along the fluid path that can be used, for example, for washing of the sample and any bound or unbound components thereof. The assay membrane can also be optionally treated to adjust the sample properties, such as, for example, by pH level or viscosity. Additional reagents can be located or applied on or inside the assay membrane. Example optional reagents added to the assay membrane can be any combination of, but not limited to, antibodies, salts, surfactants, detergents, macromolecules, small molecules, small molecules nanoparticles, microspheres, and antigens, where the reagents can be added or applied as liquid or solids to the assay membrane.

Components of the assay membrane such as the physical structure of the device described herein can be prepared from, for example, copolymers, blends, laminates, metallized foils, metallized films or metals, waxes, adhesives, or other suitable materials known to the skilled person, and combinations thereof. Alternatively, device components can be prepared from copolymers, blends, laminates, metallized foils, metallized films or metals deposited on any one or a combination of the following materials or other similar materials known to the skilled person, examples include but are not limited to paraffins, polyolefins, polyesters, styrene containing polymers, polycarbonate, acrylic polymers, chlorine containing polymers, acetal homopolymers and copolymers, cellulosics and their esters, nitrocellulose, fluorine containing polymers, polyamides, polyimides, polymethylmethacrylates, sulfur containing polymers, polyurethanes, silicon containing polymers, other polymers, glass, and ceramic materials. Alternatively, components of the device can be made with plastic, polymer, elastomer, latex, silicon chip, or metal. In one example, the elastomer can comprise polyethylene, polypropylene, polystyrene, polyacrylates, silicon elastomers, or latex. Alternatively, components of the device can be prepared from latex, polystyrene latex or hydrophobic polymers. In one example, a hydrophobic polymer can be used for the cartridge or membrane support comprising, for example, polypropylene, polyethylene, or polyester. Alternatively, components of the device can comprise TEFLON®, polystyrene, polyacrylate, or polycarbonate. Alternatively, device components can be made from plastics which are capable of being embossed, milled or injection molded or from surfaces of copper, silver and gold films upon which may be adsorbed various long chain alkanethiols. The structures of plastic which are capable of being milled or injection molded can comprise, for example, a polystyrene, a polycarbonate, a polyacrylate, or cyclo-olefin polymer. The assay membrane can also comprise an optional filter material which can be placed within and/or downstream the sample addition area to filter particulates from the sample, for example to filter or trap blood cells or particulate matter from blood so that added plasma can travel through the device.

Various configurations of diagnostic assay membranes and lateral flow assay membranes are known that can be used with the present method and prepared as described, including but not limited to variation in device dimensions, materials, porosity of the substrate, presence or absence of topographical features on the substrate, channel shape and configuration, and method of manufacturing the channel. The particular assay membrane is referred to throughout this description in terms of an exemplary embodiment, however it will be readily apparent that other device designs and possible variants of these designs could also be similarly configured. The described assay membrane 10 is particularly useful for immunoassay formats which are typically sandwich assays wherein the membrane is coated with an immobilized capture antibody or protein, sample is added, and any analyte of interest, either antigen or antibody, present in the sample binds to the immobilized capture molecule at a test region. In common immunoassays, a detecting antibody binds to antigen in the sample, an enzyme-linked secondary antibody binds to the detecting antibody or to the antigen, and a substrate in the fluid is converted by the enzyme into a detectable form.

In an automated system or analyzer, detection can be done automatically using a visualization or imaging system such as a camera or other detection system. The visualization system can further include one or more light sources emitting the same or different wavelengths of light, one or more lenses for focusing and enlargement of the test area, and one or more optical filters for eliminating or selecting specific wavelengths of light. The imaging device can also comprise one or more photodiode, photoresistor, phototransistor, camera, focal plane array, spectrometer, hall effect sensor, photomultiplier tube, antennas, and electrode.

FIG. 2A is an illustration of the results area of an example assay membrane, such as the one shown in FIG. 1. In results area 18 there are two test regions, 24a and 24b, and control region 26. In advance of adding running buffer to the assay membrane the two test regions, 24a and 24b, and optionally also the control region 26, which were marked with a localization label, are visualized using a visualization system. Alternatively, other variations of localization label addition and binding as well as variations on imaging modalities can be used. In all cases the visualization system pre-localizes the test region(s) and also optionally the control region(s) on the assay membrane in the results area 18 by imaging a localization label to enable the analyte of interest to be found in the same region by imaging an analyte label using the same or different imaging modality. The dashed lines indicate the contours of the location of line pre-localization of the region of interest (ROI) around each of the test and control regions before the analyte of interest is detected, with a margin around each line to ensure that the luminosity of the analyte of interest bound to the test region is properly read. In a preferable embodiment the margin of the ROI is less than half of the distance from one region of interest to a different region of interest. In another preferable embodiment the margin on the side of each region of interest is less than 0.5 mm, less than 0.3 mm, or less than 0.2 mm.

FIG. 2B is an illustration of one test region in a region of interest on a flow assay membrane. The region of interest (ROI) 28 indicated by the dashed lines is shown as between the predetermined contours at the location of the pre-localized test region 24 which was detected with a visualization system and imaging modality to localize the localization label on the test region with a reasonable margin. After the assay is run, the visualization system in an analyzer can locate the region of interest on the flow assay membrane based on automated membrane alignment, and optionally with the addition of external reference features such as cartridge feature location, cartridge port or opening alignment with the imaging system, and/or other location markers such as relative locations of the regions of interest to other detectable regions of interest such as the control region or other test regions. The intensity of the test region 24 at the pre-localized ROI on the assay membrane can then be measured after the assay is run to determine the presence or absence of the analyte of interest in the sample. In addition, the intensity of the test region at the pre-localized location can be used to quantitatively determine the concentration or concentration range of the analyte of interest in the sample based on the detected intensity of the localization label as detected at the test region 24.

The localization label has a molecular property used for detection of the labeling species that can be imaged in advance of running the assay or after running the assay, and that differentiates the region of interest where the localization label has been applied from the background of the assay membrane. The differentiating molecular nature or property of the localization label can be, for example, wavelength or color, frequency, phase, amplitude, intensity, delay time, energy, fluorescence lifetime, refractive index, reflectance, absorbance, emissivity, transmittance, polarization, dispersion, and scattering. The localization label molecular properties can be observed using a detector or imaging modality which may have any combination of, but not limited to, one or more photodiodes, photoresistors, phototransistors, cameras, focal plane arrays, spectrometers, hall effect sensors, photomultiplier tubes, antennas, and electrodes. If needed, an external stimulus to stimulate the molecular property can be used, for example any combination of but not limited to temperature variation, mechanical force, electromagnetic wave, chemical reaction, biochemical reaction, radiation, electron transfer, light filtration, light polarization, and light splitting.

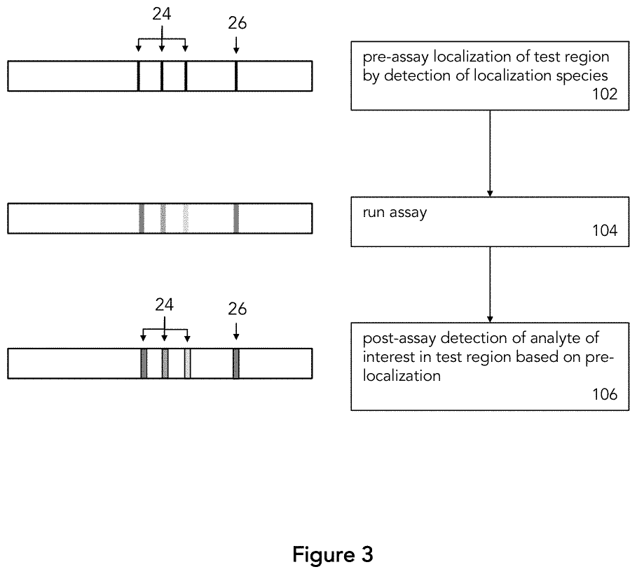

FIG. 3 illustrates one general method for pre-localization of regions of interest on an assay membrane by applying the localization label and immobilized binding species to the assay membrane during manufacture. In the assay membrane shown there are three test regions 24 and one control region 26, however it is understood that the pre-labeled assay membrane can have one or more test regions and preferably at least one control region. In the first step, the assay membrane is visualized using a visualization system to provide a pre-assay localization of the test and optionally the control regions and identification of the region(s) of interest on the assay membrane by detection of the localization label 102. This process can be generalized to other methods of localization where one or more result areas are searched for any shape, number, or size of regions of interest. After the locations of the test regions have been determined and the regions of interest identified, a running buffer is added to the assay membrane to run the assay 104. Pre-run analysis is then used to locate the one or more test region(s) 24 in the results area on the assay membrane in the region(s) of interest after the assay is run, which is used later to reduce the amount of background noise captured post-run by the imaging or visualization device. By pre-locating the test region at the region(s) of interest the visualization system can be programmed to ignore any area surrounding the test region(s), which are sources of background noise, and focus on the region(s) of interest where the immobilized binding species is localized. Background noise increases the uncertainty of any system, especially near the limit of detection. In assay runs with low signal, distinguishing the background noise from regions of interest can be near impossible and can result in inconclusive assay results. Post-assay detection of the analyte label at the test regions in the results area of the assay membrane can then done based on the line pre-localization 106 providing a meaningful signal even with a low signal result of the analyte of interest. The signal intensity of each region of interest, i.e. at each of the test and control regions, can thus be measured by limiting the use of the collected image data to the location of each region of interest, optionally with a reasonable margin, to reduce the noise contribution to the signal from the region of interest.

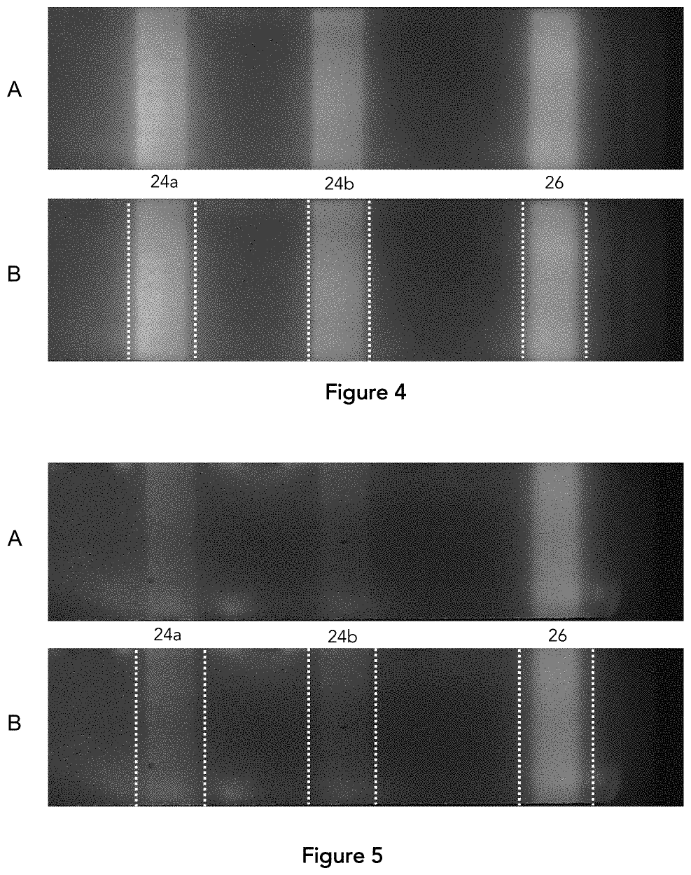

FIG. 4 is an example high signal result from a lateral flow assay membrane assay after an assay run. Image A shows the results area from the lateral flow assay with fluorescence imaging of bound Europium to two test regions 24a, 24B and a control region 26. The images were taken using a CMOS (complementary metal-oxide-semiconductor) camera, a UV LED and the Europium reporter. Prior to the assay run the same results area was imaged to pre-localize the contours of the regions of interest as shown by the dashed lines in image B, and the localization label on the test and control regions was imaged. After the assay run the image processing of the area at the test and control regions could be restricted to the contour areas of the region of interest identified by the presence of localization label before the assay and limited to the area between each set of dashed lines. In a high signal result where the test and control regions are bright and discernable in post-assay imaging the line pre-localization can be useful for excluding extraneous signal evidenced by gradient shading adjacent the control and test regions.

FIG. 5 is an example low signal result from a lateral flow assay membrane assay. Image A shows the results area from the lateral flow assay with fluorescence imaging of bound Europium to two test regions 24a, 24B and a control region 26 where the test regions are more difficult to discern due to lower contrast in the image at the regions of interest compared to background. In the low signal case, determination of the test region(s) location in an automated system can be challenging as the signal to noise ratio is high compared to background. However, using line pre-localization the signal intensity can still be meaningfully measured by restricting the quantified signal to the contour area at the region of interest around which the bound immobilized species is known to be on the results area of the assay membrane. Image B shows the same results area where the regions of interest at the test and control regions were pre-localized such that image analysis can be done only on the pre-localized regions of interest. In this way even low signal assay results can be meaningfully read and recorded, reducing the false negative incidence of the test. Further, signal amplification in the localized region(s) of interest can provide higher accuracy results for calculation for samples which have a low concentration analyte of the interest. Pre-localizing the test region(s) increases the limit of detection of the lateral flow assay by providing a signal measurement in the location where the immobilized species is known to be, and calibration of the optical or visualization system and data analysis method improves the accuracy of analysis of the assay results. Further, pre-determination of the location of the immobilized binding species improves high throughput automation results by limiting the data analysis to the known region of interest, thus limiting processing time and resulting in a more reliable result. The process of using a localization label is particularly useful when algorithms that search test areas for regions of interest for positive signals are used. These algorithms look for significant signal:noise changes and use these changes to identify a region of interest on an assay membrane. In this instance, any region with a high background in a negative sample run result area may falsely be labeled as the region of interest. This can also happen for very low positive sample runs. This “false localization” can increase the imprecision of a population of negative and low positive samples, and the increase in imprecision in turn decreases the limit of detection of an assay by making a low positive sample difficult to statistically distinguish from a negative sample. Using pre-localization of regions of interest ensures that image analysis is performed at the location where the bound species is known to be on the assay membrane.

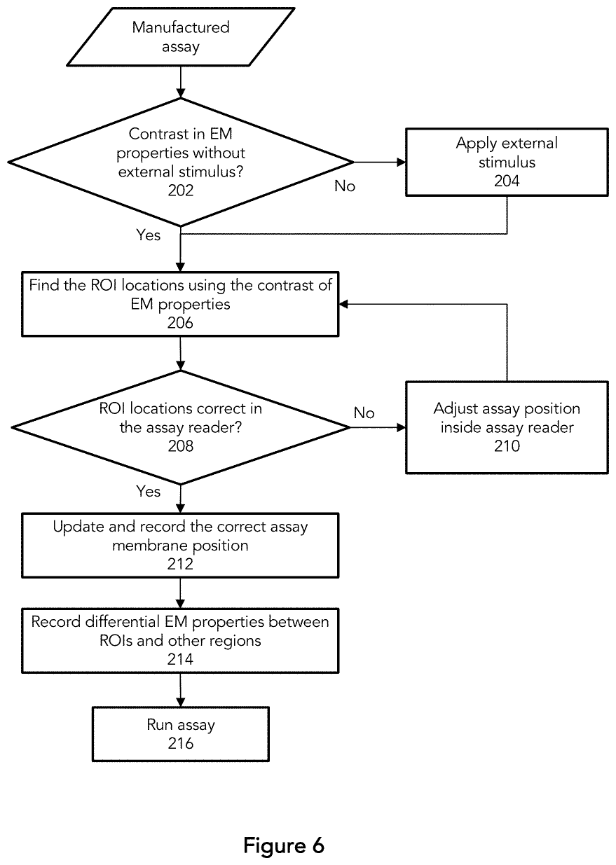

FIG. 6 is a flowchart of a method for pre-localization of a region of interest on a flow assay membrane with localization label and immobilized binding species pre-applied to the test region(s). A manufactured assay membrane necessarily has variation, and the present method reduces noise in the assay result and can normalize the variation using pre-detection of a localization species on the assay membrane before the assay is run. In an automated assay reader or analyzer, the contrast in electromagnetic or molecular property of a localization label at a region of interest is determined using an imaging device before the assay run. Molecular properties at the region(s) of interest can be observed with or without an external stimulus depending on the localization label, and the molecular property can be detected by one or more detectors. In an example, the molecular property of the localization label can be one or more of wavelength (color), frequency, phase, amplitude, intensity, delay time, energy, fluorescence lifetime, refractive index, reflectance, absorbance, emissivity, transmittance, polarization, dispersion, and scattering, and the localization label can be detected with or without excitation prior to imaging, depending on the molecular property being detected. If the contrast in molecular property of the localization label is detectable without external stimulus 202 then the ROI locations can be found using the contrast of molecular properties 206. If an external stimulus is required, as in the case of a fluorescent detectable localization species, an external stimulus is applied 204, such as a light at or near the excitation wavelength of the fluorescent detector, and the imaging device finds the ROI locations using the contrast of molecular properties 206 with stimulus. The analyzer then confirms that the ROI locations are correct in the assay reader 208, and if not found to be correct, the position of the assay membrane in the assay reader can be adjusted 210. The assay membrane can then be imaged again to localize the ROI locations on the assay membrane 208 by locating the localization label and the correct assay membrane position is recorded for that assay membrane result area 212. The method for locating positions of regions of interest can be repeated until the position is located. This step ensures that an accurate position for region of interest is located each run, and controls for the positions of the regions of interests that are impacted by, for example, inconsistent human handling, error or drift in reader components, assay manufacturing and assay assembly. Once the assay membrane position and ROI position(s) has been recorded, the differential molecular properties between ROIs and other regions are also recorded 214 and can be used for background subtraction calculation. The assay is then run 216 by adding running buffer to the upstream end of the assay membrane.

The signal from the imaging or signal detection in the analyzer is digitized and can be transformed into, for example, a vector or multi-dimensional data array. The molecular property data arrays are then processed to locate the region of interest based on the contrast between regions of interest and other regions. To find the location of test and optionally also control region(s) of interest using the molecular property of the localization label, software algorithms in the image processing system can use, for example, one or more of cropping, rotation, smoothening, color space transformation, time-frequency domain transformation, contrast enhancement, sharpening, thresholding, amplification, clipping, averaging, feature extraction, scaling, pattern recognition, projection, component analysis, wavelet transformation, filtering, algebra calculation, histogram operation, and geometric transformation. The software algorithm can also use the relative locations of and the signal intensity at regions of interest for any of the molecular properties of the localization label for signal correction after the assay has been run, which can be done using, for example, any combination of linear or non-linear algebra calculation or transformation.

An automated analyzer is preferably used to receive the assay membrane and also preferably to process the assay results including imaging and visualization or detection of regions of interest on the membrane. In one example, the analyzer can comprise a fluid dispense area comprising a sample conduit for dispensing a fluid volume onto the assay membrane, an imaging area comprising a light source for illuminating the assay membrane and an imaging device for imaging the assay membrane, and a processor for analysing image data collected from the imaging device.

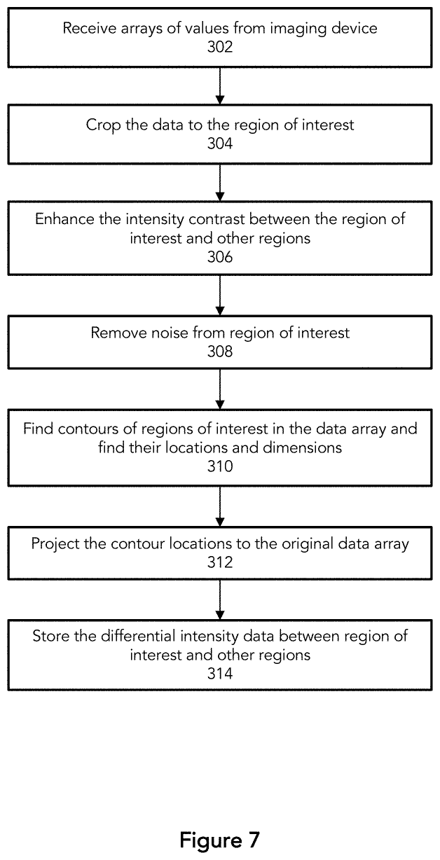

FIG. 7 is a flowchart of a visualization or detection method for pre-localization of a region of interest (ROI) on a pre-labeled assay membrane in an analyzer. The assay membrane can be pre-labeled with any localization label that can be detected by the imaging device in the analyzer or any localization label binding species that binds the localization label. The localization label can, for example, be colored or have molecular properties that can be changed with an external stimulus, such as a fluorescent species. The assay membrane is put into an imaging device or automated analyzer with an imaging device to take an image of the ROI. The imaging device can comprise, for example, one or more camera, charge-coupled device (CCD) sensor, or complementary metal oxide semiconductor (CMOS) sensor. The light or signal captured by the imaging device can be visible, infrared, of single or multiple wavelengths. A fluorescent localization label is a species that is detectable in a detection range different from the fluid sample when excited by an illumination light source and can re-emit light of a different wavelength after light excitation from the illumination source at a first wavelength. When used with a fluorescent localization label as the pre-labeling species or localization label, imaging can comprise application of an external stimulus such as light as a suitable excitation wavelength to excite the fluorescent species. Fluorescent species, such as fluorescent dyes and fluorescent proteins, can offer several advantages compared to colorimetric labels, such as greater assay sensitivity, quantitative readout, and the possibility of multiplexing for simultaneous on-site measurement of different substances from a single sample. Commonly used labels include, for example, gold nanoparticles (GNP), quantum dots, and fluorescent microspheres. The localization label applied at the region of interest has a different molecular property from the rest of regions on the assay membrane after the assay is manufactured but before the assay is run. The differences can be any and/or any combination of wavelength, color, frequency, phase, amplitude, intensity, delay time, energy, fluorescence lifetime, refractive index, reflectance, absorbance, emissivity, transmittance, polarization, dispersion, and scattering.

For pre-localization of the ROI, an imaging or detection device is used to receive array image data of the assay membrane and a processor is used to process the imaged data. The pre-localization of the test region(s) can be done either before the assay is run or after the assay is run using an imaging modality that differentiates the localization label from the analyte label. The imaging system of the analyzer includes at least one illumination light source or and at least one light receiving unit connected to a microcontroller and computer system for recording and analyzing collected imaging data. A computer capable of data analysis in the present methods comprises a processor, memory, and at least one data storage device or connection thereto. The system memory typically contains data such as data and/or program modules such as an operating system and application software that are accessible to and/or are operated on by the processor. The computer may also include other removable/non-removable, volatile/non-volatile computer storage media. The computer may be connected to the imaging device or receive data from the imaging device for processing.

Once the ROI has been imaged by the imaging device, a processor receives arrays of values from imaging device 302 and the image data is cropped to the region of interest 304. Methods such as adaptive thresholding can be used to define and crop the assay region. The intensity contrast between the region of interest and other regions 306 is then enhanced and the noise is removed as much as possible from region of interest 308. The contours of regions of interest in the data array are then found, and their locations and dimensions located 310. The analyzer finds contours in the image where the regions of interest may be present by comparing electromagnetic or pixel intensity in and around the ROI. Corrections can also be applied to increase the contrast of the image and noise is then removed from the image. The precise location and contours of the region of interest can be based on a variety of metrics such as, for example, size, aspect ratio, pixel intensity contrast, gradient slope, and relative distance of the contours. The contours that contain the location of the regions of interest are projected onto the original data array 312 and the intensity data of the region of interest is stored as well. The differential intensity data between the region(s) of interest and other regions 314 is also stored, as well as the location and size of the contours, optionally in combination with one or more other reference locations that can be additionally used to localize the regions of interest on the assay membrane.

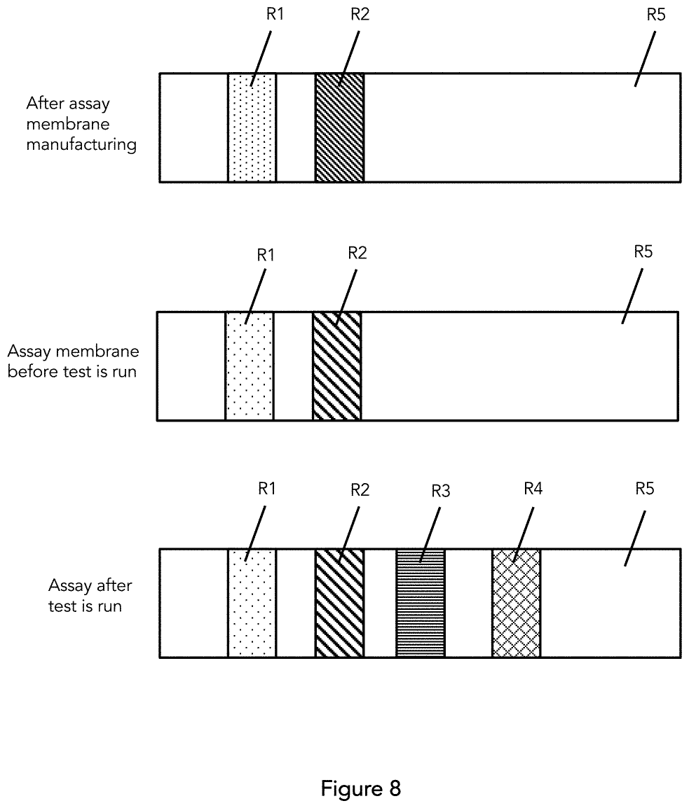

FIG. 8 is an illustration of a flow assay membrane with a localization label after manufacturing, before an assay run, and after an assay run. The pre-run localization analysis uses a proxy labeling or localization label to calibrate the location and also optionally the intensity of the test region(s) and optionally also control region(s) after run of the lateral flow assay. After assay manufacturing the regions that the optical device can locate are region of interest R1, region of interest R2, and background region R5 of the detection area. The regions of interest can exhibit different (region 1) or the same (region 2) molecular properties from the other regions (region 5) when no external excitation or stimulus is applied, and the molecular properties for the ROIs can be the same or different with the use of different localization label or combinations of localization label, or different concentrations thereof. The molecular property of the localization label can be detected by one or multiple detectors or detector combinations of, for example, photodiodes, photoresistors, phototransistors, cameras, focal plane arrays, spectrometers, hall effect sensors, photomultiplier tubes, antennas, and electrodes. The detected signals are then digitized and transformed into a vector and/or multi-dimensional data array. If needed, external excitations or stimulus can be applied by any combinations of temperature variation, mechanical force, electromagnetic wave, chemical reaction, biochemical reaction, radiation, electron transfer, filtration, polarization, and light splitting. With external excitation or stimulus, the molecular properties in ROIs (region 1-2) are different from other background regions (region 5). The molecular properties in ROIs can be the same (region 2) or different (region 1) from the situation without external excitation or stimulus, and can be the same or different from each other.

The molecular property data arrays are processed to locate the ROIs based on the contrast between ROI and other regions. Between the assay membrane manufacture date and the time the assay is run, degradation, aging, fading, and chemical and biological changes can cause a change in localization label as well as the binding species in the assay which can affect the signal measured at the different regions. Further, after the lateral flow array is run the imaging of each region will have again changed compared to the pre-assay region of interest R1, region of interest R2, and background of detection area R5. In addition, other test and control regions may be visible in the test area of the assay membrane R3, R4 where there had been no application of localization label during manufacturing but where imaging can detect a change in signal intensity after the assay run. Such regions of interest can also be localized after the assay run based on their known relative location from the regions of interest that had been pre-localized with localization label (R1/R2). In particular, the manufacture of assay membranes with multiple lines where only one or only two lines have pre-labeling may also be useful in localizing the other lines after the assay run based on the distance from one line to a labeled line or an unlabeled line. During the deposition of binding agent on the assay membrane, the quantity of the localization label can be proportional to the immobilized binding reagent at the region of interest, thus the signal from the localization label can be used as a proxy for concentration obtained from its molecular properties and can be used to calculate the concentration of immobilized binding species using a proportionality constant. Calibration of effective concentration immobilized binding species on the assay membrane to determine the concentration of analyte of interest in the sample can thereby also be done based on the differential intensity of each region of interest before and after the assay run. In particular, the signal intensity of the molecular signal detected from the ROI on the assay membrane is related to the concentration of reagents on the lateral flow assay membrane through a proportionality constant. The proportionality constant can be used to calibrate the amount or concentration of bound reagent on the test region and/or control region through calculating the signal intensity of the localization label at the ROI.

After the assay test is run, the optical properties of each region of interest can also change. In one example, the molecular properties in the ROI may change based on the external excitations and/or stimulus used for signal reading. For example, the molecular properties in region 1 after running the assay can be the same or different from other regions (region 5), which are different from before running the assay, and different from without external excitation or stimulus. The molecular properties at region 2 (R2) after running the assay can be the same as other regions (such as background R5), and/or different from the molecular properties of the same region before running the assay, and the same or different as without external excitation or stimulus. Molecular properties in region 3 (R3) and region 4 (R4) after running the assay can be the same or different from other regions (R5), the same or different as before running the assay, or the same or different as without external excitation or stimulus.

After the assay is run the assay membrane is moved back to the target position in the reader by the automated analyzer and the actual ROI locations are found and compared with the target value, with optional adjustment to the assay membrane position until the alignment is acceptable for imaging and signal processing. In particular, the analyzer can move the assay to the target position in the imaging system and the analyzer can find the locations of the region of interest using the pre-run localization data. Alternatively, computer processing can adjust the image taken after the assay run to align the received image to the pre-localization ROI locations. The device then reads the assay signal and applies signal correction using pre-run molecular properties. The signal correction can be done using any or any combination of linear or non-linear algebra calculation or transformation. The assay signal is then read and recorded, optionally with applied external stimulus, and a signal correction is applied using the previously recorded differential molecular properties. The signal correction can be done using any or any combination of linear and/or non-linear algebra calculation and/or transformation. In this example, the signal data collected for regions 1-4 can be used to indicate either a positive or negative reaction for the test. Regions 1, 3, and 4 can also be used as a control signal when region 2 is used as a test region. After the post-run analysis is complete, the device will output results.

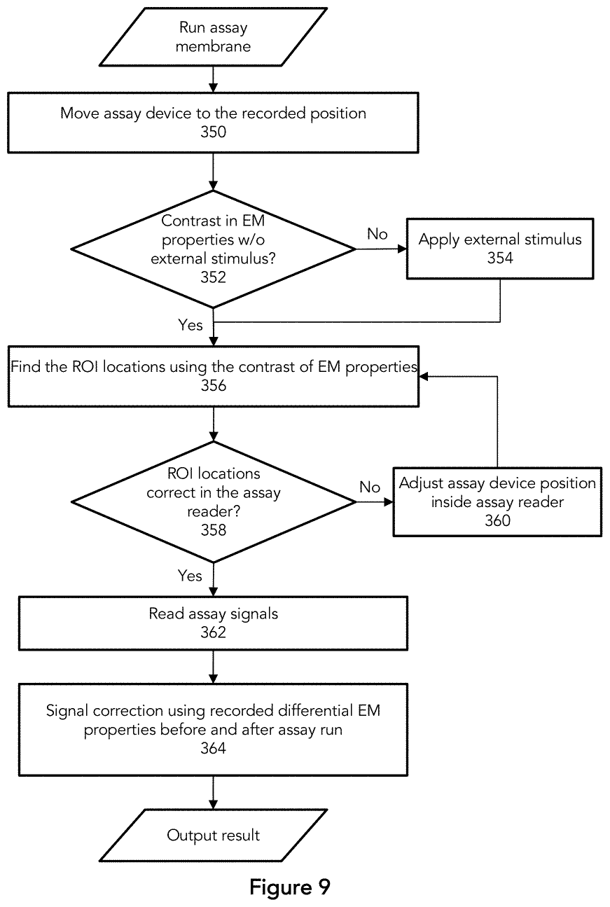

FIG. 9 is a flowchart of a method for detection of signal at a region of interest in an automated analyzer after test region pre-localization and run of the assay by addition of a running buffer. After the lateral flow assay test has been completed, the regions of interest i.e. the test and region(s), are located using the pre-labeling information and data recorded for the assay membrane. To read the assay results the assay membrane is moved to the recorded position in the imaging device 350 of the analyzer. If the contrast in molecular properties requires an external stimulus 352 then an external stimulus 354 is applied, such as appropriate illumination. If no external stimulus or illumination is needed and the contrast is sufficient then none is required. The ROI locations on the assay membrane are then found using the contrast of molecular properties 356. The analyzer then refers to the stored ROI locations and queries whether the newly found ROI locations are correct in the assay reader 358. If not, the assay membrane position inside the assay reader is adjusted 360 and the ROI locations are re-found using the contrast of molecular properties 356 to ensure that the location of the assay membrane is correct. If the positioning is correct, then the assay signals at the ROI are read 362 and signal correction is used to record the differential molecular properties using information related to molecular properties recorded before the test and after the assay run 364. The result is then output for reading and reporting.

A variety of techniques can be used in the method of detection of a region of interest (ROI) post-localization. The pre-localization method consists of two parts: a pre-localization analysis to localize the test region(s) and a post-run analysis to determine the presence and/or amount of analyte of interest bound at the test region and optionally also to verify the control region. The data processing method shown for ROI locating is based on the contrast in molecular signal at the location of the localization label compared to the background of the assay membrane. In one technique this is done using light illumination at a particular wavelength and light intensity to detect the contrast. ROIs such as those at test region and control region can also have one or more binding species, such as immobilized antibodies, in addition to the localization label, and the localization label can be deposited together with test region and control region antibodies or in a separate step. When the localization label is deposited onto the assay membrane in the same process and/or same solution as the deposited testing reagents (e.g. test region antibodies), the quantity of the localization labeling species at each ROI will be proportional to the testing reagents on the assay. In particular, a more intense contrast in molecular signal of the localization label at the ROI indicates that more binding species have also been deposited in the same location. Thus, the amount of localization label at the ROI can be used as an indication of the amount of test reagent(s), and the recorded difference in observable molecular properties (such as light intensity values in the above example) can be used to correct for the assay signal reading after the assay run. As such, the contrast in molecular signal can be indicative of the concentration of immobilized binding species as the ROI. This step can improve the assay accuracy by compensating for the variation in assay manufacturing and different readers. Once the assay membrane has been prepared with the immobilized binding species and localization label at the ROI the assay membrane can then be loaded with sample and running buffer and the ROIs can be found after the assay run using the pre-localization determination for the same assay membrane.

For location of the ROI (pre-localization) before running the assay, the localization label can be detected, for example, with a white light source and an RGB camera, or a light source capable of sufficient differentiation of the localization label compared to the background of the assay membrane. Other visualization techniques can also be used, such as, for example, fluorescence, to contrast the molecular properties of the localization label at the test regions compared to background. The contrast can then be used to generate a data array with different light intensities, indicating the relative location of the ROIs on the assay membrane. In both cases of fluorescence detection as well as visible light detection, data processing methods are similar. In one example of data processing, the ROI is digitized to an array of intensity values. The array can then be extracted and cropped to reduce data size. The data can also be cropped to the assay region by adaptive thresholding or image thresholding. The intensity contrast between the ROI and other regions can then be enhanced and separated from other regions. Noise areas can also be removed, for example by dilation and erosion. The contours in the data array can then be found, as well as their locations and dimensions, and the ROI positions and sizes can be determined by filtering by, for example, contour location, size, aspect ratio, and/or relative distance. The contour locations are then projected to the original data array.

In an example, if the detection conjugate bound to the immobilized binding species comprises a europium label, excitation of the ROI after assay run by a 365 nm light source will generate a fluorescent signal at about 635 nm at the pre-localized ROI. The differential intensity data between ROIs and other regions is also stored and the analysis outputs the assay ROI location on the assay membrane. Other optical improvements and data analysis techniques that can be used to render the data more precise or accurate could be applied. The ROI position determination process can also be repeated in a closed loop until a satisfactory location can be obtained. This iterative step ensures the accurate position of the ROIs inside the reader, which can be highly variable due to, for example, inconsistent human handling, error/drift in reader components, assay manufacturing, and assay assembly. After the assay test, if the localization label is washed away the immobilized binding species remaining on the assay membrane at the ROI can be detected using molecular contrast in combination with the pre-localized locations of the ROIs. Pre-localization of the ROIs is especially useful in the case where there is little or no molecular contrast visible at the test regions after the run, where pre-localization can provide the location contours of the pre-localized immobilized binding species to better differentiate the signal at the pre-localized ROIs.

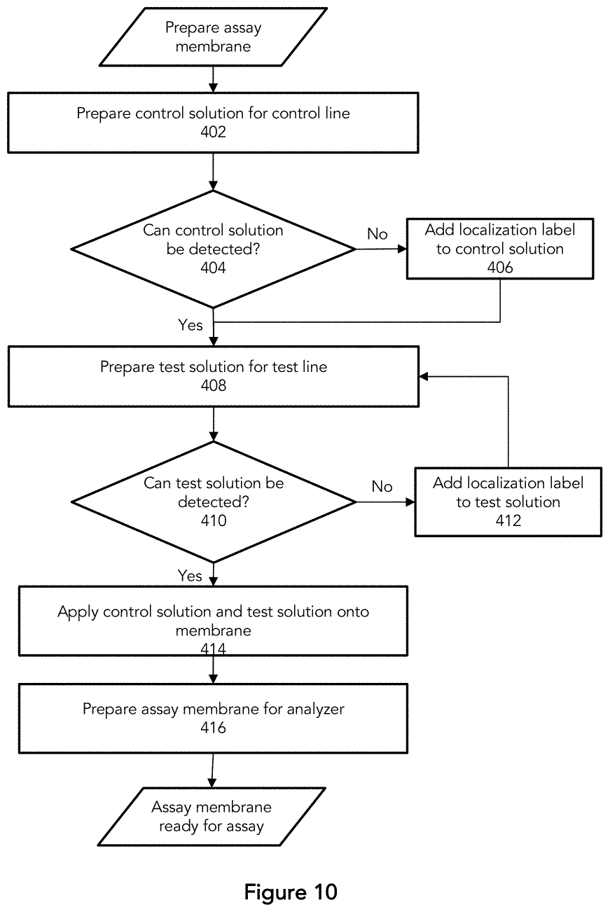

FIG. 10 is a flowchart of a method for manufacturing an assay membrane with a localization label for pre-localization of a region of interest. In the manufacture of assay membranes, detectors, binding agents, and bound species are applied to the membrane in an industrial process, often sprayed to large sheets of assay membrane. To mass manufacture lateral flow assay membranes, the prepared membrane sheets are then cut into the desired size and placed into a protective cartridge for transport, handling, and assay running. The manufacturing process of lateral flow membranes is necessarily imprecise, and minor variations are introduced in, for example, membrane size, concentration of applied reagents and materials, quality or age of applied reagents, and amount of applied reagents and materials assembled between assay membranes, as well as in their placement inside each cartridge. Uncontrollable variations are thereby present in the manufacturing of assay membranes, which can lead to uncertainty in the assay results, especially when the variations cannot be corrected or normalized. These minor variations can also have a detectable and significant effect on test results, and can significantly change the results during quantitative investigations. During manufacture of pre-labeled assay membranes, a control solution is prepared for application to the control region 402. If the control solution comprises a detectable species that has molecular properties whose contrast can be detected 404 then the detectable species in the control solution can act as the localization label. Otherwise, an additional localization label can be optionally added to the control solution 406 for creating the labeled control region(s). The test solution is prepared for the test region 408 in a similar manner, whereby if the test solution comprises a detectable species that can be detected without the addition of a localization label 410 no additional localization label is required, otherwise a localization label or localization label binding species is added to the test solution 412. By pre-localizing the each bound and/or labeled species the location, concentration, and amount of sprayed species can be detected either prior to the assay run or after the assay run and prior to imaging the analyte of interest, and the results with contoured test region can be used for more accurate quantitation of the analyte of interest in the sample after the membrane has been exposed to a sample and the assay developed. In addition, starting concentration of detectors, and other bound species can be incorporated into quantitative calculations of sample concentration to more accurately discern concentrations of species of interest in the sample. The control solution and test solution are then applied to the assay membrane 414, which is often done using spray application. The assay membrane is then prepared for use in the analyzer 416, which often involves stabilization and/or protection of the assay membrane in a protective cartridge.

In another embodiment of the present invention, the assay membrane can be pre-labeled at the regions of interest with an immobile localization label that does not interfere with binding of the analyte of interest to the immobilized binding species or detection of the detectable species at the region of interest. This embodiment uses a localization label that has a molecular property that is detectable in a different imaging modality or, for example, at a different wavelength than the detection range of the detectable species that indicated binding of the analyte of interest to the immobilized binding species at the region of interest. In one example of this embodiment, sample and running buffer can be added to the assay membrane to develop the assay and allow any analyte of interest in the sample to bind to the immobilized binding species and detectable species at the region of interest. After the assay is run a first imaging method which can detect the presence of the immobilized localization label at the ROI is used to locate the ROI and assign the contours of the ROI. A second and different imaging method is then done to detect the presence of the detectable species inside the contours of the ROI to determine the binding of any analyte of interest in the ROI. For example, the first imaging method to image the localization label can use red light illumination and a digital camera, and the second imaging method can use fluorescent illumination where the detectable species is one that has fluorescent molecular properties. In this method the assay membrane only needs to be moved by the analyzer into an imaging area one time, after the assay run, if the analyzer has two modes of imaging and detection, one for each of the localization label and the detectable species.

### Example 1

An experiment was done to demonstrate pre-localization of test and control regions and visualization thereof in a lateral flow assay. Brilliant blue FCF was added as a localization label to a control solution and to a test solution and the solutions were applied to an assay membrane at a control region and test region, respectively. Prior to running the assay, an optical system took images of the test region and control region using white light through a detection window before the lateral flow assay. The brilliant blue FCF was used as a localization label to indicate the locations of the test and control regions and the location data was stored. Sample containing an analyte of interest and a running buffer was then added to the membrane and the assay was run. A fluorescence probe that binds to the analyte of interest was also present in the assay. After the lateral flow assay run the optical system took a second image of the detection area under UV light (to excite the fluorescence probe), and the reflectance values were recorded. The analysis algorithm, rather than searching for the test and control regions using intensity data, used the pre-localization, pre-run localization data to locate the contours of the test region and control region (ROIs) based on prior identification of the location of the brilliant blue FCF. The reflectance values in the region that were located pre-run were then used to calculate the concentration of the analyte of interest.

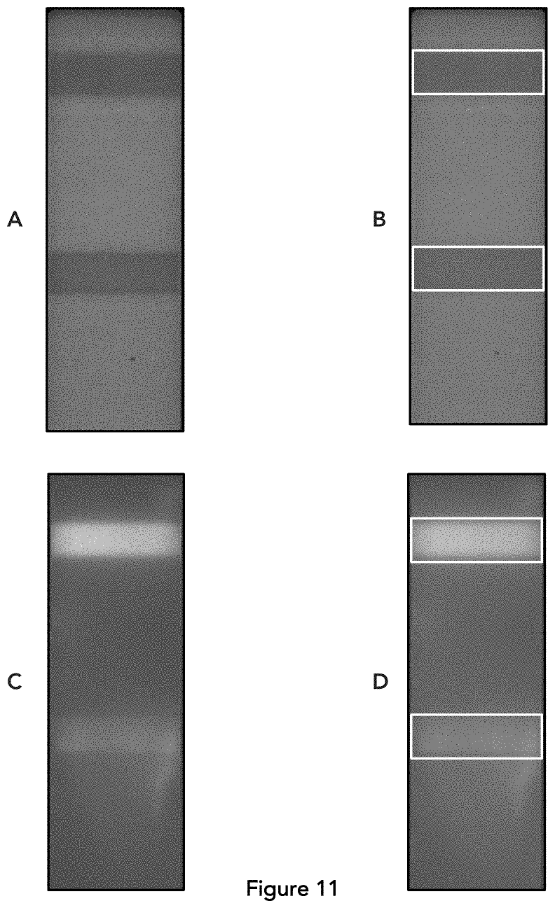

FIG. 11 is a panel of assay membranes with regions of interest during the pre-localization and post-localization method as described in this experiment. Panel A is an image of a pre-localized test region (bottom) and control region (top) with the localization label contrast to background shown as observed under white light. Panel B is an image of the pre-localized test and control regions with localization margins identified based on molecular contrast under white light of the localization label and background. Panel C is an image of a post-assay lateral flow membrane with test region (bottom) and control region (top) taken under UV light showing the presence of bound fluorescent label. It is notable that the bottom band or test band is very diffuse, with blurry margins. Panel D is an image of post-assay test and control regions with pre-localized margins indicating the location of the immobilized binding species.

Test region localization on an assay membrane provides a boundary region for automated analysis such that the results region where binding of the species of interest can be analyzed with confidence. Localizing the test binding region, either before the assay run or afterwards, and doing so in a way that differentiates the imaging results of the localization label from those of the binding species, enables more accurate measurement of the bound species of interest at the binding region while excluding noise in the surrounding non-binding region. It has been found that this method enables lower threshold concentration measurement of the analyte of interest in a test sample and results in fewer erroneous automation errors which can provide false negative and false positive results when the concentration of analyte of interest in the test sample is below a noise threshold.

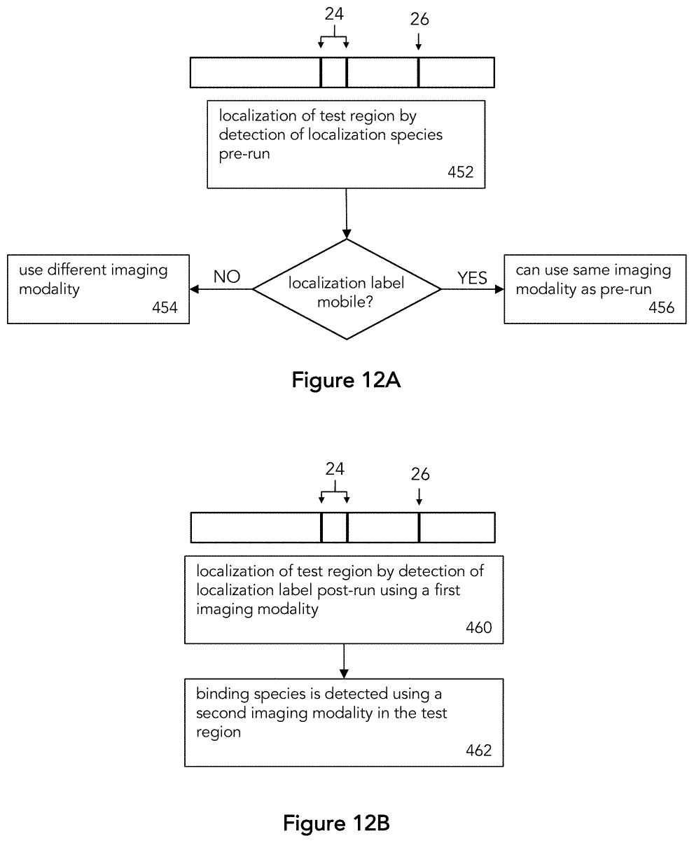

FIG. 12A is a flowchart of a method for test region localization on an assay membrane using a pre-run localization of the region of interest. In this method the assay membrane is pre-labeled with a localization label which is applied at the test region 24, preferably during assay membrane manufacture. In one method, the assay membrane can be prepared by mixing the localization label and immobilized binding species together in a test solution and applying the solution to the assay membrane in a test region or test region. Application of the test solution to the test region or test region is preferably done with spraying. After application to the membrane the localization label can be either unbound to the membrane and flow away with the running buffer during the assay, or can be bound to the test region and remain in place after the assay run. In use with this type of localization system, the test region in the region of interest can be localized by detection of the pre-applied localization label before the assay run. This is done by imaging the region of interest and localizing the localization label on the test region 452 or test region. The test region 24 is also preferably localized in relation to one or more features on the membrane, such as another test region or control region 26 or other recognizable feature that can be imaged, or to a recognizable feature on the membrane cartridge or housing. Using a reference feature can further assist in aligning the assay membrane during imaging of the test region after the assay has been run with sample. If the localization label is mobile, meaning the localization label is unbound to the membrane and soluble in the running buffer, the same imaging modality can be used to image the assay membrane after the assay run 456, provided that there is no interference with the detected signal at the test region from the localization label. If the localization label is bound or anchored to the assay membrane and remains at the test region after the assay run, a different imaging modality that images only the bound species 454 can be used, provided that the localization label does not interfere with anchored binding species at the binding modality selected for the binding species and analyte of interest. The two different imaging modalities can vary along, for example but not limited to, imaging wavelength, fluorescence excitation wavelength, fluorescence emission wavelength, and light excitation or emission polarization.

FIG. 12B is a flowchart of a method for test region localization on an assay membrane using more than one imaging modality in a post-run localization method. In this method the region of interest has two immobilized capture species: a first immobilized binding species to capture the localization label, and a second immobilized binding species to capture the molecule of interest and the binding species label. In an immunoassay, the first immobilized binding species is a first capture antibody for capturing the localization label where the localization label comprises a binding antigen attached to a detectable label, and the second immobilized binding species is a second capture antibody for capturing the species of interest and a mobile detection antibody having a bound detectable species. The localization label and detectable species which binds to the species of interest are each detectable at a different and non-interfering imaging modality such that the presence of each in the same region can be detected independently. Using this method, the localization label can be either applied to the region of interest during assay manufacture such that it is anchored to the test region or can be applied with the sample and/or the running buffer and have a conjugate that binds to an anchored species at the test region. In both cases the localization label can be localized at the test region after the assay run and imaged using a different imaging modality than that used to detect the species of interest. After running buffer and sample are added to the assay membrane and the assay is developed, the localization of the test region in the region of interest can be done by detection of the localization label post-run using a first imaging modality 460. A second imaging modality is then used to detect the binding species label which binds to the analyte of interest, if present in the sample, in the region of interest 462. In an immunoassay the binding species label forms a sandwich with the antigen (molecule of interest) and an immobilized capture antibody at the test region. It is understood that the second imaging modality is not significantly affected by the presence of the localization label in the same region as the binding species detected and that the presence of the localization label does not interfere with detection of the binding species in the second imaging modality. In one example use of the method, the localization label and the binding species label can be added together with the sample and/or the running buffer and flow together at the test region in the region of interest. In another example the localization label and the binding species label can be added sequentially after the sample containing the binding species or analyte of interest. In either case two bound species at the test region, one to bind the localization label and the second to bind the binding species label are non-competitive and do not affect the binding of the other species.

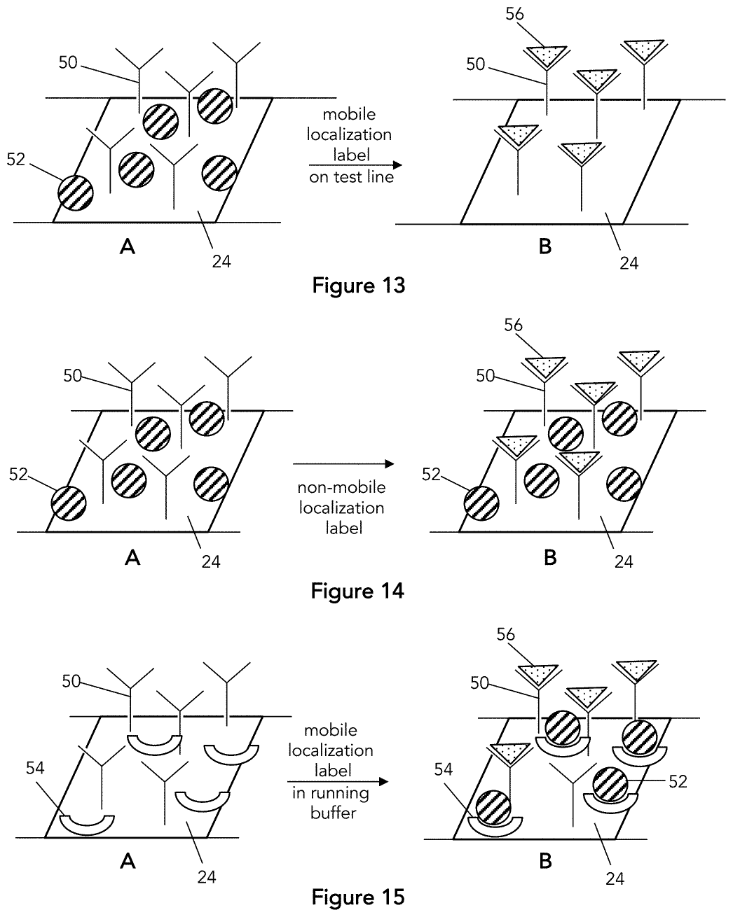

FIG. 13 shows an assay membrane test region pre-localization with an assay membrane having a mobile localization label pre-applied to the test region. In this variation the localization label 52 and immobilized binding species 50 are applied to the assay membrane at a test region 24 during manufacture of the assay membrane (A). The immobilized binding species 50 is preferably a capture antibody that has a high affinity to the analyte of interest which the assay membrane is designed to capture and measure from a test sample. In this variation the localization label 52 is mobile and soluble in the running buffer in the assay such that it will be substantially removed and carried downstream from the test region 24 during the assay run, leaving the analyte of interest 56 from an applied sample bound to the immobilized binding species 50 together with an analyte label at the test region 24 after the assay run (B). This assay membrane design can be used in the present method by localizing the localization label at the test region 24 with a first imaging modality before the assay is run, and then imaging the test region 24 after the assay is run with the same or different imaging modality. Since substantially all of the localization label 52 will have been carried away from the test region 24 by the assay running buffer it is possible to use the same imaging modality to image the conjugate of the analyte of interest with analyte label bound to the immobilized binding species 50.

FIG. 14 shows an assay membrane test region pre-localization with an assay membrane having a non-mobile localization label pre-applied to the test region. In this variation the localization label 52 and immobilized binding species 50 are applied to the assay membrane at a test region 24 during manufacture of the assay membrane (A). The immobilized binding species 50 is preferably a capture antibody that has a high affinity to the analyte of interest which the assay membrane is designed to capture and measure from a test sample. In this variation the localization label 52 is non-mobile and immobilized or bound to the test region 24 during the assay run such that it is substantially not soluble in the assay running buffer. After the running buffer is added to the assay membrane together with the analyte of interest in a sample solution, any analyte of interest 56 together with an analyte label will bind to the immobilized binding species 50 at the test region 24 (B). This assay membrane design can be used in the present method by localizing the localization label at the test region 24 with a first imaging modality before or the assay is run, and then imaging the test region 24 after the assay is run with a different imaging modality to image the analyte label bound to the analyte of interest 56 and immobilized binding species 50. It is noted that it is also possible to image the test region 24 before the assay run (A) using a first imaging modality, and then after the assay run (B) using the same imaging modality and interpret the signal of the analyte of interest by subtracting the pre-run signal from the post-run signal.

FIG. 15 shows an assay membrane test region localization with an assay membrane having a localization label binding species pre-applied to the test region and a localization label in the assay running buffer. In this variation a localization label binding species 54 that binds a mobile localization label 52 is applied to the test region 24 during manufacture of the assay membrane along with the immobilized binding species 50 which binds an analyte of interest in the sample (A). The immobilized binding species 50 is preferably a capture antibody that has a high affinity to the analyte of interest which the assay membrane is designed to capture and measure from a test sample. In this variation the localization label 52 is added to the sample solution and/or running buffer, and upon reaching the test region 24 during the assay run binds to the localization label binding species 54 (B). Concurrently the analyte of interest in a sample solution will bind to the immobilized binding species 50 at the test region 24 (B). This assay membrane design can be used in the present method by localizing the localization label at the test region 24 with a first imaging modality after the assay is run to localize the test region using the localization label 52, and then imaging the same test region 24 with a different imaging modality to image the analyte label bound to the analyte of interest 56 and immobilized binding species 50.

In cases where the localization label is applied to the test region along with the immobilized binding species during manufacture, it is also possible to gauge the age and state of degradation of the otherwise invisible immobilized binding species by imaging the localization label and comparing the expected signal to the actual signal obtained. It has been found that deterioration of the localization label at the test region can provide an indication of overall assay membrane sensitivity and can be an indication of assay membrane quality.

All publications, patents and patent applications mentioned in this specification are indicative of the level of skill of those skilled in the art to which this invention pertains and are herein incorporated by reference. The reference to any prior art in this specification is not, and should not be taken as, an acknowledgement or any form of suggestion that such prior art forms part of the common general knowledge.

The invention being thus described, it will be obvious that the same may be varied in many ways. Such variations are not to be regarded as a departure from the scope of the invention, and all such modifications as would be obvious to one skilled in the art are intended to be included within the scope of the following claims.

1. A method for localizing an analyte of interest on a test region of an assay membrane comprising:
imaging a localization species in the test region, the localization species having a molecular property that, upon imaging, differentiates the test region from a background of the assay membrane;
determining contours of the test region by imaging the localization label and the background around the region of interest and comparing intensity of the background of the assay membrane to intensity at the region of interest; and
imaging an analyte of interest inside the contours of the test region after exposing the assay membrane to a running buffer to run the assay, the analyte of interest bound to a detectable analyte label and an immobilized binding species at the test region.

2. The method of claim 1, wherein imaging the localization label in the test region is performed prior to running the assay, and further comprising, before imaging the analyte of interest:
applying a sample comprising the analyte of interest to the assay membrane; and
applying a running buffer to the assay membrane to run the assay.

3. The method of claim, wherein the localization label is at least one of an organic dye, inorganic dye, fluorescent molecule, phosphorescent molecule, radiating molecule, and colored bead.

4. The method of claim 1, wherein the localization label is brilliant blue FCF, prussian blue, quinoline yellow WS, gold nanoparticles, europium nanoparticles, Cu doped zinc sulfide, glass beads, carbon nanotubes, HgTe quantum dots, phthalocyanine, or a combination thereof.

5. The method of claim 1, wherein imaging the localization label comprises exposing the region of interest to an external stimulus to image a contrast between the localization label and the background.

6. The method of claim 5, wherein the external stimulus is white light or ultraviolet light.

7. The method of claim 5, wherein the localization label comprises a fluorescent species, and the external stimulus comprises a light source in an absorbance band of the fluorescent species.

8. The method of claim 1, wherein the molecular property of the localization label is one or more of wavelength, frequency, phase, amplitude, intensity, delay time, energy, fluorescence lifetime, refractive index, reflectance, absorbance, emissivity, transmittance, polarization, dispersion, and scattering.

9. The method of claim 1, wherein the localization label is applied to the test region before manufacturing, and the localization label is soluble in the running buffer and washed away from the test region during the assay.

10. The method of claim 1, wherein the localization label on the assay membrane is in an amount proportional to the immobilized binding species at the region of interest in a proportionality constant, the method further comprising using the proportionality constant to calculate a concentration of analyte of interest in the sample.

11. The method of claim 1, wherein the assay membrane is a lateral flow assay membrane.

12. The method of claim 1, carried out in an automated analyzer.

13. A method for manufacturing an assay membrane comprising:
applying a localization label to a test region on an assay membrane, the localization label having a molecular property that, upon imaging, differentiates the test region from a background of the assay membrane; and
applying an immobilized binding species to the test region of interest on the assay membrane, the immobilized binding species configured to bind with an analyte of interest,
wherein the localization label does not interfere with binding of the immobilized binding species to the analyte of interest during an assay run.

14. The method of claim 13, wherein the localization label is soluble in assay running buffer.

15. The method of claim 13, further comprising mixing the localization label and the immobilized binding species in a test solution and applying the test solution to the assay membrane during manufacturing.

16. The method of claim 13, wherein the localization label and the immobilized binding species are present in a known ratio at the region of interest.

17. A lateral flow assay device comprising:
a sample addition area;
a results area downstream the sample addition area comprising at least one test region and at least one control region, the test region comprising an immobilized binding species and a localization label, the localization label having a molecular property that, upon imaging prior to assay run, differentiates a region of interest around the test region from a background in the results area.

18. The device of claim 17, wherein the localization label on the test region is in an amount proportional to the immobilized binding species.

19. The device of claim 17, wherein the localization label is soluble in assay running buffer and washed away from the results area by the running buffer during the assay run.

20. The device of claim 17, wherein the molecular property of the localization label is one or more of wavelength, color, frequency, phase, amplitude, intensity, delay time, energy, fluorescence lifetime, refractive index, reflectance, absorbance, emissivity, transmittance, polarization, dispersion, and scattering.
